(19) United States
(12) Patent Application Publication
(10) Pub. No.: US 2026/0128138 A1
(43) Pub. Date: May 7, 2026

(54) AI-Based System and Method for Generating Enhanced Radiology Reports

(71) Applicant: Alexander Davis, Oxnard, CA (US)

(72) Inventor: Alexander Davis, Oxnard, CA (US)

(21) Appl. No.: 18/935,609

(22) Filed: Nov. 3, 2024

(51) Int. Cl. G16H 15/00; G16H 10/60; G16H 20/10; G16H 30/20; G16H 30/40; G16H 50/20; G16H 50/30; G16H 50/70; G16H 70/20

(52) U.S. Cl. CPC G16H 15/00; G16H 10/60; G16H 20/10; G16H 30/20; G16H 30/40; G16H 50/20; G16H 50/30; G16H 50/70; G16H 70/20

(57) ABSTRACT

The present invention relates to an AI-based system and method for generating enhanced radiology reports. The system comprises a database for storing multimodal patient data, a natural language processing (NLP) module for extracting clinical information, and a machine learning module for correlating the clinical information with radiology images to identify diagnostic insights. An AI-based report generation module analyzes the images and clinical information to generate a preliminary report, which is refined based on radiologist input. The generated report is then integrated into the patient's electronic health record. The system employs techniques such as multimodal deep learning, active learning, explainable AI, and federated learning to enhance diagnostic accuracy, capture expert feedback, provide transparency, and enable multi-institutional collaboration. The invention aims to improve the accuracy, efficiency, and value of radiology reporting in patient care.

### BACKGROUND OF THE INVENTION

### Field of Invention

The various aspects discussed herein relate to systems and methods for generating enhanced radiology reports using artificial intelligence.

### Description of Related Art

Radiologists analyze medical images to diagnose various health conditions. However, conventional radiology reporting workflows face several challenges. First, radiologists often lack access to a patient's complete clinical history, which can provide valuable context for interpreting images. Second, manually analyzing complex images is time-consuming and prone to human variability and errors. Third, radiology reports are often unstructured and may lack key information needed by referring physicians for optimal treatment planning.

Accordingly, there is a need in the art for an AI-based radiology reporting system that integrates multimodal patient data, generates comprehensive diagnostic insights, and produces structured reports that facilitate clinical decision-making. Such a system would improve the accuracy, efficiency, and value of radiology services in patient care.

### BRIEF SUMMARY OF THE INVENTION

This summary is provided to introduce a selection of concepts, in a simplified format, that are further described in the detailed description of the invention. This summary is neither intended to identify key or essential inventive concepts of the invention nor is it intended for determining the scope of the invention.

The present invention provides systems and methods for generating enhanced radiology reports using artificial intelligence (AI). In one aspect, the system comprises a database for storing multimodal patient data, including radiology images, blood test results, physical exam records, and patient-reported symptoms. A natural language processing (NLP) module extracts relevant clinical information from the patient data, which a machine learning module then correlates with the radiology images to identify diagnostic insights.

Another important embodiment incorporates biopsy results. an application-specific integrated circuit (ASIC) for an artificial neural network connected to the computer memory device, the ASIC comprising: a plurality of neurons organized in an array, wherein each neuron comprises a register, a processing element and at least one input, and a plurality of synaptic circuits, each synaptic circuit including a memory for storing a synaptic weight, wherein each neuron is connected to at least one other neuron via one of the plurality of synaptic circuits configured, configured to

An AI-based report generation module analyzes the radiology images in conjunction with the correlated clinical information to generate a preliminary report. This report includes an AI-suggested diagnosis and visual highlights of key regions of interest on the images. The preliminary report is presented to a radiologist for review and modification. The radiologist's input is used to update the machine learning module, enabling continuous refinement of the AI system. Finally, a report integration module incorporates the AI-generated radiology report into the patient's electronic health record (EHR).

The AI system may employ a multimodal deep learning architecture that integrates natural language processing of clinical notes, computer vision analysis of radiology images, and structured data from labs and vital signs. An active learning framework selectively prompts radiologists for input on uncertain cases to efficiently capture expert feedback. Explainable AI techniques provide human-interpretable visual and textual explanations of the factors influencing the AI's diagnostic predictions, enhancing transparency and trust.

Additionally the present invention may include a clinical decision support module that provides evidence-based diagnostic and treatment recommendations, a reinforcement learning framework that automatically adapts the AI models based on radiologist feedback and patient outcomes, and a federated learning module enabling secure multi-institutional collaboration without data sharing. A question-answering system can automatically extract relevant information from the EHR to provide radiologists with contextual insights. Predictive analytics may leverage the AI-generated insights and longitudinal EHR data to identify high-risk patients and recommend proactive interventions.

The present invention solves the problems of incomplete clinical context, time-consuming manual image analysis, and unstructured reporting associated with conventional radiology workflows. By integrating multimodal data, generating comprehensive diagnostic insights, and producing structured reports, the AI system improves the accuracy, efficiency, and clinical utility of radiology services. This enhanced radiology reporting system has the potential to streamline diagnostic processes, increase productivity, reduce errors and variability, and ultimately lead to better patient outcomes and reduced healthcare costs across a wide range of radiology practices and healthcare institutions.

It is intended that embodiments of the present invention include but not be limited to different types or radiology, among them rojection (plain) radiography, Fluoroscopy, Computed tomography, ultrasound, Magnetic resonance imaging, and different types of Nuclear medicine like Positron emission tomography (PET).

Additional features and advantages of the invention will be set forth in the description which follows. These and other features of the present invention will become more fully apparent from the following description, or may be learned by the practice of the invention as set forth hereinafter.

### BRIEF DESCRIPTION OF THE DRAWINGS

The various exemplary embodiments of the present invention, which will become more apparent as the description proceeds, are described in the following detailed description in conjunction with the accompanying drawings, in which:

FIG. 1 is a block diagram illustrating a system for generating an enhanced radiology report according to an embodiment.

FIG. 2 illustrates a radiology report generation user interface according to an embodiment of the system shown in FIG. 1.

FIG. 3 is a flow diagram illustrating a method for generating an enhanced radiology report using the system depicted in FIG. 1 via the user interface shown in FIG. 2.

### DETAILED DESCRIPTION

In the following detailed description of the preferred embodiments, reference is made to the accompanying drawings, which form a part hereof and show, by way of illustration, specific embodiments in which the invention may be practiced. It is to be understood that other embodiments may be used and structural or logical changes may be made without departing from the scope of the present invention. The following detailed description, therefore, is not to be taken in a limiting sense, and the scope of the present invention is defined by the appended claims.

The following description is provided as an enabling teaching of the present systems, and/or methods in its best, currently known aspect. To this end, those skilled in the relevant art will recognize and appreciate that many changes can be made to the various aspects of the present systems described herein, while still obtaining the beneficial results of the present disclosure. It will also be apparent that some of the desired benefits of the present disclosure can be obtained by selecting some of the features of the present disclosure without utilizing other features.

Accordingly, those who work in the art will recognize that many modifications and adaptations to the present disclosure are possible and can even be desirable in certain circumstances and are a part of the present disclosure. Thus, the following description is provided as illustrative of the principles of the present disclosure and not in limitation thereof.

The terms “a” and “an” and “the” and similar references used in the context of describing a particular embodiment of the present invention (especially in the context of certain claims) are construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein.

All systems described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. No language in the specification should be construed as indicating any non-claimed element essential to the practice of the application. Thus, for example, reference to “an element” can include two or more such elements unless the context indicates otherwise.

As used herein, the terms “optional” or “optionally” mean that the subsequently described event or circumstance can or cannot occur, and that the description includes instances where said event or circumstance occurs and instances where it does not.

The word or as used herein means any one member of a particular list and also includes any combination of members of that list. Further, one should note that conditional language, such as, among others, “can,” “could,” “might”, or “may” unless specifically stated otherwise, or otherwise understood within the context as used, is generally intended to convey that certain aspects include, while other aspects do not include, certain features, elements and/or steps. Thus, such conditional language is not generally intended to imply that features, elements and/or steps are in any way required for one or more particular aspects or that one or more particular aspects necessarily include logic for deciding, with or without user input or prompting, whether these features, elements and/or steps are included or are to be performed in any particular aspect.

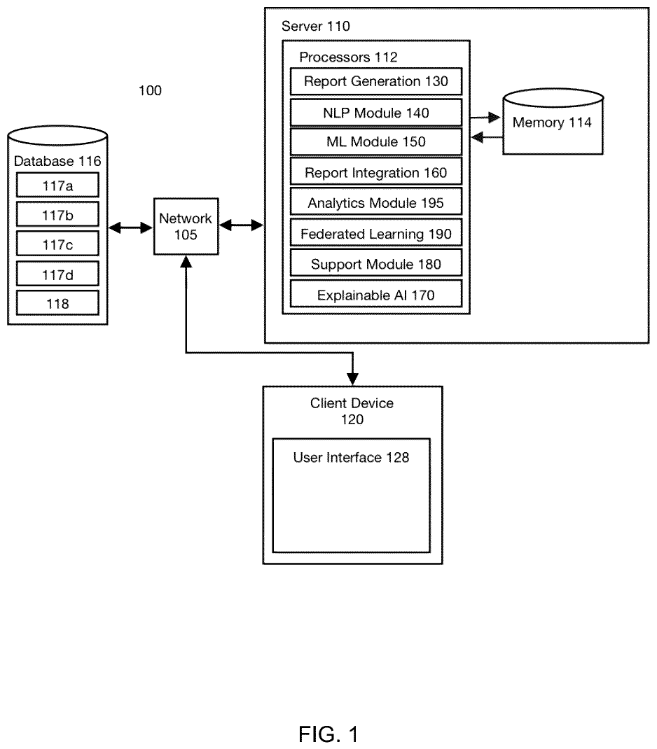

FIG. 1 is a block diagram illustrating a system 100 for generating an enhanced radiology report according to an embodiment. In one embodiment, the system 100 comprises a server 110 communicatively coupled to a client device 120 via a network 105, wherein said network 105 may include, by way of example and not limitation, the Internet, a local area network (LAN), a wide area network (WAN), or any other suitable wired or wireless communication network.

In some embodiments, the server 110 includes one or more processors 112, such as central processing units (CPUs), microprocessors, or any other suitable computing devices, and a memory 114, such as random access memory (RAM), read-only memory (ROM), or any other suitable storage medium. In one embodiment, the memory 114 is configured to store computer-executable instructions that, when executed by the processor(s) 112, cause the server 110 to implement an artificial intelligence (AI) based radiology report generation module 130, a natural language processing (NLP) module 140, a machine learning module 150, and a report integration module 160. According to an embodiment, the server 110 is operably connected to a database 116 configured to store patient data, wherein the patient data may include, but is not limited to, one or more of radiology images 117a (e.g., X-rays, computed tomography (CT) scans, magnetic resonance imaging (MRI) scans, ultrasound images, positron emission tomography (PET) scans), blood test results 117b (e.g., complete blood count (CBC), metabolic panel, lipid profile), physical examination records 117c (e.g., vital signs, clinical findings), and patient-reported symptoms 117d (e.g., pain, fatigue, appetite changes).

In one embodiment, the NLP module 140 is configured to extract relevant clinical information from the patient data stored in the database 116 using techniques such as tokenization, part-of-speech tagging, named entity recognition, and dependency parsing. According to an embodiment, the machine learning module 150 is trained using supervised, unsupervised, or semi-supervised learning algorithms to correlate the extracted clinical information with the radiology images 117a to identify relationships and generate diagnostic insights. In some embodiments, the machine learning module 150 may employ various architectures such as convolutional neural networks (CNNs), recurrent neural networks (RNNs), or graph neural networks (GNNs) to learn hierarchical features and patterns from the multimodal data.

In some embodiments, the machine learning module 150 utilizes a deep learning architecture based on convolutional neural networks (CNNs) for analyzing the radiology images 117a. In this embodiment, the CNN consists of an input layer, multiple convolutional and pooling layers, and fully connected layers. The input layer accepts radiology images with the dimensions of 512×512 pixels. The convolutional layers apply 64 filters of size 3×3 with a stride of 1, followed by ReLU activation and max pooling with a 2×2 window and stride of 2. The output of the final convolutional layer is flattened and passed through two fully connected layers with 128 and 64 neurons, respectively.

The CNN can be trained using a dataset of 100,000 radiology images, split into 80% training data and 20% validation data. In some embodiments, the model can be optimized using stochastic gradient descent with a learning rate of 0.01 and a batch size of 32.

In another embodiment, the NLP module 140 employs a transformer-based architecture for extracting relevant clinical information from patient data. The transformer model consists of an embedding layer, multiple self-attention layers, and a final classification layer. The input data, such as clinical notes, are tokenized and converted into word embeddings of size 256. The self-attention layers have 8 attention heads and a hidden size of 512. The final classification layer outputs the probability of each clinical entity (e.g., symptoms, medications, procedures) being present in the input data. In some embodiments, the NLP module 140 is trained on a corpus of 500,000 clinical notes, with 90% used for training and 10% for validation. In another embodiment, the model can be optimized using the Adam optimizer with a learning rate of 0.001 and a batch size of 16.

In one embodiment, the AI-based radiology report generation module 130 is configured to:
(i) analyze a radiology image 117a in conjunction with the correlated clinical information from the machine learning module 150 using computer vision techniques such as segmentation, object detection, and classification;
(ii) generate a preliminary radiology report based on the analysis, wherein the preliminary report may optionally include an AI-generated diagnosis (e.g., presence or absence of a specific medical condition, severity grade) and visual highlights of regions of interest on the radiology image 117a using techniques such as heat maps, bounding boxes, or overlay graphics;
(iii) receive radiologist input via a user interface 128 (e.g., a graphical user interface (GUI), a voice user interface (VUI), a gesture-based interface) on the client device 120 (e.g., a desktop computer, a laptop, a tablet, a smartphone) modifying or confirming the preliminary radiology report; and
(iv) update the machine learning module 150 based on the radiologist input using techniques such as reinforcement learning, active learning, or incremental learning, thereby continuously improving the performance and generalizability of the AI models.

In some embodiments, the AI-based radiology report generation module 130 integrates the outputs from the machine learning module 150 and the NLP module 140 using a multimodal fusion technique. The image features extracted by the CNN and the clinical entities identified by the transformer model are concatenated and passed through a series of fully connected layers with 256, 128, and 64 neurons, respectively. The final layer outputs the probability of each finding and recommendation being included in the radiology report. The report generation module 130 can be trained on a dataset of 50,000 radiology reports, with 80% used for training and 20% for validation. In one embodiment, the model can be optimized using the Adam optimizer with a learning rate of 0.0001 and a batch size of 8. After training for 20 epochs, the report generation module can achieve a BLEU score of 0.85 on the validation set.

In one embodiment, the AI-based radiology report generation module 130 employs a multimodal deep learning architecture that integrates natural language processing of clinical notes, computer vision analysis of radiology images, and structured data from lab results and vital signs to generate a holistic diagnostic assessment. In some embodiments, the multimodal architecture may leverage techniques such as attention mechanisms, cross-modal fusion, or multi-task learning to effectively combine the heterogeneous data types and capture their interactions, thereby enabling comprehensive analysis of patient data.

According to an embodiment, the machine learning module 150 may incorporate an active learning framework configured to selectively prompt radiologists via the user interface 128 for input on informative and uncertain cases, thereby optimizing efficiency of capturing expert feedback for continuous improvement of the AI system 100. In some implementations, the active learning framework may employ techniques such as uncertainty sampling, query-by-committee, or expected model change to identify the most valuable instances for annotation

According to one embodiment, the report integration module 160 is configured to integrate the AI-generated radiology report into a patient's electronic health record (EHR) 118 using standards such as Health Level Seven (HL7), Fast Healthcare Interoperability Resources (FHIR), or Digital Imaging and Communications in Medicine (DICOM). In one embodiment, the EHR 118 is stored in the database 116.

In another embodiment, the system 100 may further include an explainable AI module 170 configured to generate human-interpretable visual and textual explanations of key factors influencing the AI-generated diagnostic predictions, thereby enhancing transparency and building trust with radiologists and patients. The explainable AI module 170 may employ techniques such as, by way of example and not limitation, feature attribution, counterfactual analysis, or concept activation vectors to identify the salient image regions, clinical variables, or learned representations contributing to the AI decisions.

In some embodiments, the report integration module 160 may apply natural language generation techniques such as rule-based templates, sequence-to-sequence models, or transformer architectures to automatically summarize key findings and recommendations from the AI-generated radiology report into a concise format for inclusion in the patient's EHR 118. The generated summaries may be configured to adapt to the preferences and writing styles of different radiologists or institutions based on learning from historical reports.

Alternatively, the system 100 may further include a clinical decision support module 180 that integrates the AI-generated radiology insights with evidence-based guidelines, relevant clinical trials, and similar past cases, thereby providing radiologists with contextually relevant diagnostic and treatment recommendations. The clinical decision support module 180 may employ techniques including but not limited to case-based reasoning, knowledge graphs, or recommender systems to retrieve and rank the most pertinent supporting information.

In one embodiment, the machine learning module 150 may incorporate a reinforcement learning framework that automatically adapts hyperparameters and architectures of underlying deep learning models based on a reward signal derived from radiologist feedback and patient outcomes. The reinforcement learning framework may employ techniques such as policy gradients, Q-learning, or actor-critic methods to learn optimal strategies for dynamically adjusting the AI models to different clinical contexts and user preferences.

According to an embodiment, the system 100 optionally includes a federated learning module 190 configured to enable the AI system to securely learn from decentralized patient data across multiple institutions without requiring data sharing, thereby enhancing generalizability and robustness of the diagnostic models. The federated learning module 190 may employ techniques comprising secure aggregation, differential privacy, or homomorphic encryption to protect patient privacy while enabling collaborative learning.

According to another embodiment, the federated learning module 190 enables collaborative learning across multiple institutions without the need for data sharing. Each participating institution trains a local copy of the AI model using their own patient data. The model parameters are then sent to a central server, where they are averaged to create a global model. The updated global model is then distributed back to the participating institutions for further local training. This process is repeated for multiple rounds until convergence. The federated learning module employs differential privacy techniques to ensure that no sensitive patient information is leaked during the parameter averaging process. The model architecture and hyperparameters are similar to those used in the centralized AI system, with the addition of secure aggregation and noise injection mechanisms to maintain data privacy.

In some embodiments, the NLP module 140 may employ a question-answering architecture configured to automatically extract clinically relevant information from the patient's EHR 118 to answer radiologists' queries and provide contextual insights during a diagnostic process. The question-answering architecture may leverage techniques such as information retrieval, reading comprehension, or knowledge distillation to efficiently locate and synthesize relevant evidence from the unstructured EHR data.

According to an embodiment, the system 100 may further comprise a predictive analytics module 195 coupled to the AI-generated radiology insights and the longitudinal EHR data, wherein the predictive analytics module 195 is configured to predict patient trajectories, identify high-risk individuals, and recommend proactive interventions for improving outcomes and reducing costs. In another embodiment, the predictive analytics module 195 may employ techniques including, but not limited to, time-series modeling, survival analysis, or Markov decision processes to forecast disease progression, treatment response, or adverse events.

In one embodiment, the various modules and components of the system 100 operate in concert to provide a comprehensive AI-based solution for enhancing radiology workflows and decision-making. By way of example and not limitation, the system 100 may integrate multimodal data analysis, active learning from expert feedback, explainable AI techniques, and seamless EHR integration, thereby empowering radiologists with robust diagnostic support while ensuring transparency and continuous improvement of the underlying AI models. In some embodiments, the modular architecture of the system 100 allows for flexible deployment, scalability, and customization to meet the diverse needs of different healthcare organizations and radiology practices.

The explainable AI module 170 employs Grad-CAM to highlight salient image regions influencing the AI's predictions. Specifically, it computes the gradients of the predicted class score with respect to the final convolutional layer activations and uses them to weight the importance of each activation map. The weighted activation maps are then combined and upsampled to the original image size to create a heat map visualizing the salient regions.

For example, given a 1024×1024 chest X-ray, the CNN generates a feature vector of size 1024, which is concatenated with the RNN's output (a feature vector of size 256 encoding clinical note information). The concatenated vector is used to predict pneumonia with 90% confidence. Grad-CAM generates a heat map highlighting the lower right lung, consistent with the consolidation visible in the X-ray.

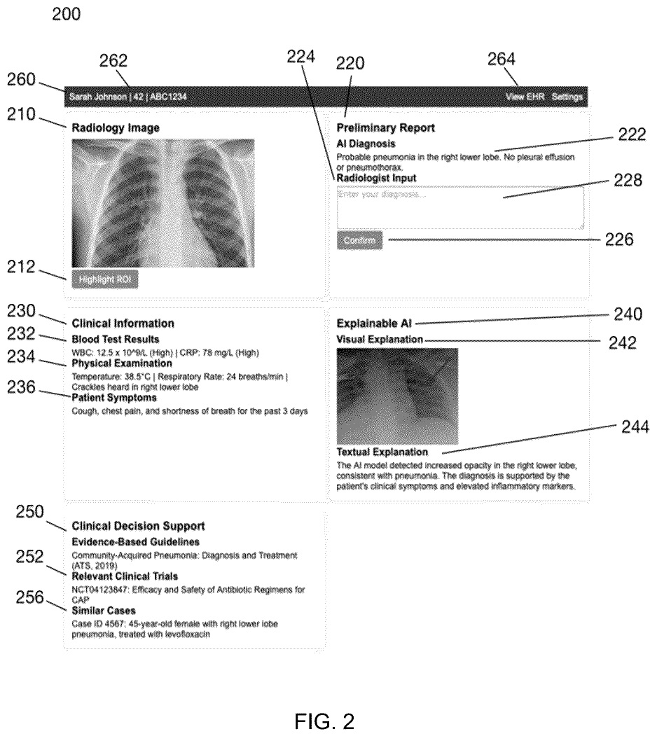

FIG. 2 illustrates a radiology report generation user interface 200 according to an embodiment of the system 100 shown in FIG. 1, wherein the radiology report generation user interface 200 enables a user to interact with the AI-based radiology report generation module 130 via the client device 120.

As shown in FIG. 2, the radiology report generation user interface 200 comprises a radiology image display area 210 configured to display one or more radiology images 117a, wherein the radiology image display area 210 includes a region of interest highlighting button 212 configured to visually highlight regions of interest identified by the AI-based radiology report generation module 130.

In one embodiment, adjacent to the radiology image display area 210 is a preliminary report display area 220 that presents a preliminary radiology report generated by the AI-based radiology report generation module 130, wherein the preliminary report display area 220 comprises an AI-generated diagnosis section 222 and a radiologist input section 224, and wherein the radiologist input section 224 is configured to allow a radiologist to modify or confirm the AI-generated diagnosis using a diagnosis confirmation button 226 and a diagnosis input field 228.

In another embodiment, disposed below the preliminary report display area 220 is a clinical information display area 230 that presents relevant clinical information extracted by the NLP module 140, wherein the clinical information display area 230 includes a blood test results section 232, a physical examination records section 234, and a patient-reported symptoms section 236.

According to an embodiment, the radiology report generation user interface 200 further comprises an explainable AI section 240 configured to provide human-interpretable visual and textual explanations of key factors influencing the AI-generated diagnostic predictions, wherein the explainable AI section 240 includes a visual explanation display 242 and a textual explanation display 244.

In some embodiments, disposed at the bottom of the radiology report generation user interface 200 is a clinical decision support section 250 configured to integrate the AI-generated radiology insights with evidence-based guidelines, relevant clinical trials, and similar past cases, wherein the clinical decision support section 250 includes an evidence-based guidelines display 252, a relevant clinical trials display 254, and a similar cases display 256.

As depicted in FIG. 2, the radiology report generation user interface 200 also comprises a header section 260 that displays patient information 262, such as the patient's name, age, and identification number, wherein the header section 260 further includes a navigation menu 264 with options configured to access other system functions, such as viewing the patient's EHR 118 or adjusting system settings, thereby enhancing the user's ability to navigate and interact with the system 100.

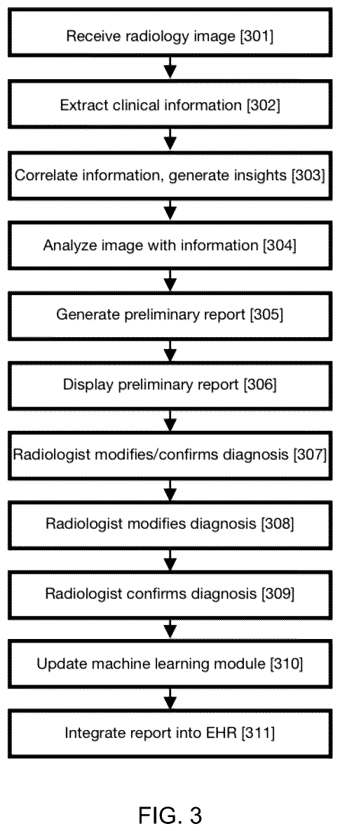

FIG. 3 is a flow diagram illustrating a method 300 for generating an enhanced radiology report using the system 100 depicted in FIG. 1 via the user interface 200 shown in FIG. 2. In one embodiment, the method 300 comprises a series of steps performed by the system 100, wherein said steps are represented by rectangular elements connected by unidirectional arrows indicating the flow and sequence of the steps.

In one embodiment, the method 300 begins at step 301, wherein the AI-based radiology report generation module 130 receives a radiology image 117a for analysis. The flow then proceeds to step 302, wherein the NLP module 140 extracts relevant clinical information from the patient data stored in the database 116, said clinical information comprising blood test results 117b, physical examination records 117c, and patient-reported symptoms 117d.

Next, at step 303, the machine learning module 150 correlates the extracted clinical information with the radiology image 117a to identify relationships and generate diagnostic insights. The flow then moves to step 304, wherein the AI-based radiology report generation module 130 analyzes the radiology image 117a in conjunction with the correlated clinical information from the machine learning module 150.

In another embodiment, at step 305, the AI-based radiology report generation module 130 generates a preliminary radiology report based on the analysis performed in step 304. Optionally, the preliminary report includes an AI-generated diagnosis and visual highlights of regions of interest on the radiology image 117a.

The method 300 proceeds to step 306, wherein the preliminary radiology report is displayed on the radiology report generation user interface 200 via the user interface 128 on the client device 120. In this embodiment, the preliminary report is presented in the preliminary report display area 220, with the AI-generated diagnosis shown in the AI-generated diagnosis section 222 and the radiology image 117a with highlighted regions of interest displayed in the radiology image display area 210.

At decision step 307, the radiologist reviews the preliminary report and determines whether to modify or confirm the AI-generated diagnosis. If the radiologist chooses to modify the diagnosis, the flow moves to step 308, wherein the radiologist provides input via the diagnosis input field 228 in the radiologist input section 224. Alternatively, if the radiologist chooses to confirm the diagnosis, the flow proceeds to step 309, wherein the radiologist confirms the AI-generated diagnosis using the diagnosis confirmation button 226.

From either step 308 or step 309, the flow proceeds to step 310, wherein the AI-based radiology report generation module 130 updates the machine learning module 150 based on the radiologist input received in steps 308 or 309, thereby enabling continuous improvement of the AI system 100.

Finally, at step 311, the report integration module 160 integrates the AI-generated radiology report into the patient's electronic health record (EHR) 118, thereby completing the method 300.

The embodiments described herein are given for the purpose of facilitating the understanding of the present invention and are not intended to limit the interpretation of the present invention. The respective elements and their arrangements, materials, conditions, shapes, sizes, or the like of the embodiment are not limited to the illustrated examples but may be appropriately changed. Further, the constituents described in the embodiment may be partially replaced or combined together.

Another important embodiment incorporates biopsy results. According to this embodiment, an application-specific integrated circuit (ASIC) for an artificial neural network connected to the computer memory device, the ASIC comprising: a plurality of neurons organized in an array, wherein each neuron comprises a register, a processing element and at least one input, and a plurality of synaptic circuits, each synaptic circuit including a memory for storing a synaptic weight, wherein each neuron is connected to at least one other neuron via one of the plurality of synaptic circuits configured, is configured to make a prediction or classification about biopsy results based on some input training data of negative and positive biopsy results, which can be labeled as such. The algorithm will produce an estimate about a pattern in the data.

An error function evaluates the prediction of the model. If there are known examples, an error function can make a comparison to assess the accuracy of the model.

A model optimization process then occurs. If the model can fit better to the data points in the training set, then weights are adjusted to reduce the discrepancy between the known example and the model estimate. The algorithm will repeat this “evaluate and optimize” process, updating weights autonomously until a threshold of accuracy has been met.

Supervised learning in particular uses a training set to teach models to yield the desired output. This training dataset includes inputs and correct outputs, which enables the model to learn over time. The algorithm measures its accuracy through the loss function, adjusting until the error has been sufficiently minimized.

Thus, through the computer-implemented process described above, the present invention can improve its ability to predict and detect cancers, by using biopsy results and making a comparison to radiology images.

A blind or double-blind diagnostic process can be used in embodiments, where the radiologist and AI make independent assessments before seeing each other's results, would add a layer of accountability and safety. This could greatly reduce over-reliance on AI and maintain the radiologist's active role.

Tracking how well individual radiologists perform compared to the AI can provide valuable data, ensuring both the AI and radiologist are held accountable and can be improved. This could lead to continuous performance assessment for the radiologists, as well as feedback that helps the AI improve in cases where the human assessment was superior.

What is claimed is:

1. A system for generating an enhanced radiology report, said system comprising:
a database configured to store patient data, wherein said patient data includes one or more of radiology images, blood test results, physical examination records and patient-reported symptoms;
a natural language processing (NLP) module configured to extract relevant clinical information from the patient data stored in said database;
a machine learning module comprising an application-specific integrated circuit (ASIC) for an artificial neural network connected to the database, the ASIC comprising: a plurality of neurons organized in an array, wherein each neuron comprises a register, a processing element and at least one input, and a plurality of synaptic circuits, each synaptic circuit including a memory for storing a synaptic weight, wherein each neuron is connected to at least one other neuron via one of the plurality of synaptic circuits trained to correlate said extracted clinical information with said radiology images to identify relationships and generate diagnostic insights;
an artificial intelligence (AI) based radiology report generation module configured to:
i. analyze a radiology image in conjunction with said correlated clinical information from said machine learning module;
ii. generate a preliminary radiology report based on said analysis, wherein said preliminary report optionally includes an AI-generated diagnosis and visual highlights of regions of interest on said radiology image;
iii. receive radiologist input modifying or confirming said preliminary radiology report; and
iv. update said machine learning module based on said radiologist input; and
v. a report integration module configured to integrate said AI-generated radiology report into a patient's electronic health record.

2. The system of claim 1, wherein said AI-based radiology report generation module employs a multimodal deep learning architecture that integrates natural language processing of clinical notes, computer vision analysis of radiology images, and structured data from lab results and vital signs to generate a holistic diagnostic assessment.

3. The system of claim 1, wherein said machine learning module incorporates an active learning framework that selectively prompts radiologists for input on informative and uncertain cases, whereby optimizing efficiency of capturing expert feedback for continuous improvement of the AI system.

4. The system of claim 1, further comprising:
an explainable AI module that generates human-interpretable visual and textual explanations of key factors influencing the AI-generated diagnostic predictions, thereby enhancing transparency and building trust with radiologists and patients.

5. The system of claim 1, wherein said report integration module applies natural language generation techniques to automatically summarize key findings and recommendations from the AI-generated radiology report into a concise format for inclusion in the patient's EHR.

6. The system of claim 1, further comprising:
a clinical decision support module that integrates the AI-generated radiology insights with evidence-based guidelines, relevant clinical trials, and similar past cases, thereby providing radiologists with contextually relevant diagnostic and treatment recommendations.

7. The system of claim 1, wherein said machine learning module incorporates a reinforcement learning framework that automatically adapts hyperparameters and architectures of underlying deep learning models based on a reward signal derived from radiologist feedback and patient outcomes.

8. The system of claim 1, further comprising:
a federated learning module that enables the AI system to securely learn from decentralized patient data across multiple institutions without requiring data sharing, thereby enhancing generalizability and robustness of the diagnostic models.

9. The system of claim 1, wherein said NLP module employs a question-answering architecture that can automatically extract clinically relevant information from the patient's EHR to answer radiologists' queries and provide contextual insights during a diagnostic process.

10. The system of claim 1, further comprising:
a predictive analytics module that leverages the AI-generated radiology insights, along with longitudinal EHR data, to predict patient trajectories, identify high-risk individuals, and recommend proactive interventions for improving outcomes and reducing costs.

11. A method for generating an expanded radiology report, said method comprising:
accessing, from a database, a radiology image and associated patient data, wherein said associated patient data includes at least one selected from the group consisting of blood test results, physical examination records, and patient-reported symptoms;
extracting, by a natural language processing (NLP) module, relevant clinical information from the accessed patient data;
correlating, by a machine learning module, said extracted clinical information with said radiology image to identify relationships and generate diagnostic insights;
analyzing, by an artificial intelligence (AI) based radiology report generation module, said radiology image in conjunction with said correlated clinical information;
generating a preliminary radiology report based on said AI analysis, wherein said preliminary report optionally includes a suggested diagnosis and visual highlights of regions of interest on said radiology image;
receiving radiologist input modifying or confirming said preliminary radiology report;
updating said machine learning module based on said received radiologist input; and
integrating said AI-generated radiology report into the patient's electronic health record.

12. The method of claim 11, wherein said analyzing by the AI-based radiology report generation module employs a multimodal deep learning architecture that integrates natural language processing of clinical notes, computer vision analysis of radiology images, and structured data from lab results and vital signs to generate a holistic diagnostic assessment.

13. The method of claim 11, wherein said correlating by the machine learning module incorporates an active learning framework that selectively prompts radiologists for input on informative and uncertain cases, whereby optimizing efficiency of capturing expert feedback for continuous improvement of the AI system.

14. The method of claim 11, further comprising:
generating, by an explainable AI module, human-interpretable visual and textual explanations of key factors influencing the AI-generated diagnostic predictions, thereby enhancing transparency and building trust with radiologists and patients.

15. The method of claim 11, wherein said integrating the AI-generated radiology report into the patient's electronic health record comprises:
applying natural language generation techniques to automatically summarize key findings and recommendations from the AI-generated radiology report into a concise format for inclusion in the patient's electronic health record.

16. The method of claim 11, further comprising:
providing, by a clinical decision support module, radiologists with contextually relevant diagnostic and treatment recommendations by integrating the AI-generated radiology insights with evidence-based guidelines, relevant clinical trials, and similar past cases.

17. The method of claim 11, wherein said updating the machine learning module incorporates a reinforcement learning framework that automatically adapts hyperparameters and architectures of underlying deep learning models based on a reward signal derived from radiologist feedback and patient outcomes.

18. The method of claim 11, further comprising:
securely learning, by a federated learning module, from decentralized patient data across multiple institutions without requiring data sharing, thereby enhancing generalizability and robustness of the diagnostic models.

19. The method of claim 11, further comprising:
predicting patient trajectories, identifying high-risk individuals, and recommending proactive interventions for improving outcomes and reducing costs by a predictive analytics module that leverages the AI-generated radiology insights along with longitudinal electronic health record data.

20. The method of claim 11, wherein said extracting relevant clinical information by the NLP module employs a question-answering architecture that can automatically extract clinically relevant information from the patient's electronic health record to answer radiologists' queries and provide contextual insights during a diagnostic process.
